(19) United States
(12) Patent Application Publication
(10) Pub. No.: US 2026/0355155 A1
(43) Pub. Date: Nov. 12, 2026

(54) PHARMACEUTICAL COMBINATION AND USES THEREOF FOR TREATING CANCER

(71) Applicant: Anbogen Therapeutics USA, Inc., Wilmington, DE (US)

(72) Inventors: KIEN THIAM TAN, Taipei (TW); YI CHEN LIN, Taipei (TW); TZU HSIEN YANG, Taipei (TW)

(73) Assignee: Anbogen Therapeutics USA, Inc., Wilmington, DE (US)

(21) Appl. No.: 19/576,004

(22) Filed: Mar. 24, 2026

Related U.S. Application Data
(60) Provisional application No. 63/776,300, filed on Mar. 24, 2025.

(51) Int. Cl. A61K 31/404; A61K 31/7068; A61P 35/00

(52) U.S. Cl. CPC A61K 31/404; A61K 31/7068; A61P 35/00

(57) ABSTRACT

Disclosed herein are pharmaceutical combinations comprising (E)-N-hydroxy-3-(1-(phenylsulfonyl)indolin-5-yl)acrylamide (ABT-301) and gemcitabine, and pharmaceutical kits comprising the same, and methods of using the pharmaceutical combinations in the treatment of cancer. Preclinical in vitro studies showed enhanced anti-tumor activity with the combinations described above. In vivo studies demonstrated a significant decrease in tumor growth with the combination therapy compared to gemcitabine and ABT-301 alone, suggesting potential clinical utility.

### CROSS REFERENCE

This application claims priority to, and the benefit of, U.S. Provisional Application No. 63/776,300, filed on Mar. 24, 2025, the content thereof is incorporated by reference herein.

### FIELD OF THE INVENTION

The present disclosure relates to new pharmaceutical combinations comprising (E)-N-hydroxy-3-(1-(phenylsulfonyl)indolin-5-yl)acrylamide (alternatively known as ABT-301, or MPT0E028) and gemcitabine, and pharmaceutical kits comprising the same, and to methods of using the pharmaceutical combination in the treatment of cancer.

### BACKGROUND OF THE INVENTION

Histone deacetylases (HDACs) are a class of enzymes that regulate histone acetylation and thus regulate gene expression to inhibit tumor progression. HDACs are classified according to four categories: class I (HDAC1, 2, 3, and 8); class IIa (HDAC4, 5, 7 and 9) and class IIb (HDAC6 and 10); class III (SIRT1-7); and class IV (HDAC11). They are involved in the post-translational modifications of core histone and nonhistone proteins.

These are involved in the post-translational modifications of core histone and nonhistone proteins. U.S. Pat. No. 8,846,748 discloses certain indolyl or indolinyl hydroxamate compounds as HDAC inhibitors having potent anticancer activity. Masahiro Yoshikawa et al. indicate that HDAC inhibitors prevent fibrosis in the liver, skin and lung, U.S. Pat. No. 11,278,523 B2 discloses effect of the HDAC inhibitors on treating lung fibrosis, liver fibrosis or renal fibrosis, but most of their underlying mechanisms remain to be elucidated, and suggests that TSA, an HDAC inhibitor, induces several inhibitory factors of TGF-beta 1 signals, such as Id2 and BMP-7, in human RPTEC (Masahiro Yoshikawa et al., J Am Soc Nephrol 18:58-65, 2007). Maoyin Pang and Shougang Zhuang indicate that development and progression of some chronic diseases are characterized by fibrosis, including chronic kidney disease, cardic hypertrophy and idiopathic pulmonary fibrosis (Maoyin Pang and Shougang Zhuang, The Journal of Pharmacology and Experimental Therapeutics, Vol. 355, No. 2, pp. 266-272, 2010).

As such, HDAC inhibitors (HDACi) have emerged as a promising class of drugs for treatment of cancers. Some HDACi, such as SAHA, LBH589, PXD101, MS-275, and FK228, are being examined in clinical trials for their ability to treat various solid and hematological malignancies. On the other hand, SAHA and FK228 have been approved by the U.S. Food and Drug Administration (FDA) to apply for the treatment of cutaneous T-cell lymphoma. Certain indolyl or indolinyl hydroxamate compounds as HDAC inhibitors are disclosed in U.S. patent application Ser. No. 8,846,748B2.

Nucleoside analogs constitute an important class of chemotherapeutic agents that disrupt DNA replication. Among these, gemcitabine (2′,2′-difluoro-2′-deoxycytidine), has been widely used in the treatment of various malignancies, including but not limited to pancreatic cancer, non-small cell lung cancer, breast cancer, ovarian cancer, and bladder cancer. Despite its clinical success, gemcitabine therapy is associated with limitations such as dose-limiting toxicity, rapid metabolic inactivation, and the development of resistance.

### SUMMARY OF THE INVENTION

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

Pursuant to the foregoing, the primary objective of the present disclosure is to furnish an improved therapeutic modality for the treatment of cancer. As embodied and broadly described herein, one aspect of the disclosure is directed to a pharmaceutical combination comprising a therapeutically effective amount of (E)-N-hydroxy-3-(1-(phenylsulfonyl)-indolin-5-yl)-acrylamide (ABT-301) or a pharmaceutically acceptable salt, ester, and/or solvate thereof, and a therapeutically effective amount of gemcitabine.

In certain embodiments, the (E)-N-hydroxy-3-(1-(phenylsulfonyl)-indolin-5-yl)-acrylamide is administered in a separate dosage form from the gemcitabine.

In certain embodiments, the (E)-N-hydroxy-3-(1-(phenylsulfonyl)-indolin-5-yl)-acrylamide is administered orally.

In certain embodiments, the gemcitabine is administered parenterally, intravenously, intramuscularly, subcutaneously, or intraperitoneally.

In certain embodiments, the solvate of (E)-N-hydroxy-3-(1-(phenylsulfonyl)-indolin-5-yl)-acrylamide is IPA solvate, acetone solvate, ACN solvate, MeOH solvate, NMP solvate, THF solvate, EtOAc solvate, DMAc solvate, EtOH solvate, DCM solvate, DMSO solvate, 1-4-Dioxane solvate or MIBK solvate.

As embodied and broadly described herein, one aspect of the disclosure is directed to a pharmaceutical kit for treating cancer in a subject, comprising said pharmaceutical combination.

In certain embodiments, the cancer is selected from the group consisting of pancreatic cancer, bladder cancer, colorectal cancer, breast cancer, prostate cancer, renal cancer, hepatocellular cancer, lung cancer, non-small cell lung cancer (NSCLC), ovarian cancer, cervix cancer, gastric cancer, esophageal cancer, neuroendocrine cancer, bone cancer or head and neck cancer.

As embodied and broadly described herein, one aspect of the disclosure is directed to a method for treating cancer in a subject, comprising administering to the subject a therapeutically effective amount of (E)-N-hydroxy-3-(1-(phenylsulfonyl)-indolin-5-yl)-acrylamide or a pharmaceutically acceptable salt, ester, and/or solvate thereof, and administering to the subject a therapeutically effective amount of gemcitabine.

In certain embodiments, the cancer is selected from the group consisting of pancreatic cancer, bladder cancer, colorectal cancer, breast cancer, prostate cancer, renal cancer, hepatocellular cancer, lung cancer, non-small cell lung cancer (NSCLC), ovarian cancer, cervix cancer, gastric cancer, esophageal cancer, neuroendocrine cancer, bone cancer or head and neck cancer.

In certain embodiments, the subject is a human.

In certain embodiments, the subject is a non-human mammal. More specifically, the subject is selected from the group consisting of a chimpanzee, a monkey, and a gorilla.

In certain embodiments, the subject is selected from the group consisting of a dog, a cat, a ferret, a rabbit, a guinea pig, a hamster, and a gerbil.

In certain embodiments, the subject is selected from the group consisting of a cow, a horse, a pig, a sheep, a goat, a chicken, a turkey, a duck, and a goose.

In certain embodiments, the subject is selected from the group consisting of a mouse, a rat, and a zebrafish.

In certain embodiments, the subject is selected from the group consisting of an elephant, a lion, a tiger, a bear, a deer, a whale, a dolphin, and a seal.

Accordingly, the effect of the present disclosure is that the specific combination of ABT-301 and gemcitabine can exhibit significantly stronger anti-tumor effects compared to treatment using either agent alone. This indicates the potential clinical utility of ABT-301 as a sensitizing agent in the treatment of cancer when used in combination with chemo drugs.

### BRIEF DESCRIPTION OF THE DRAWINGS

In order to make the above and other objects, features, advantages and embodiments of the present invention more obvious and understandable, the drawings are described as follows:

FIG. 1 illustrates the effectiveness of ABT-301 combined with Gemcitabine being assessed in relation to the growth of human BxPC3 tumors that were subcutaneously implanted in nude mice. FIG. 1 also illustrates changes of tumor size (A) and body weight (B) in BxPC3 tumor-bearing nude mice upon treatments.

FIG. 2 illustrates the antitumor efficacy and tolerability of ABT-301 administered alone or in combination with gemcitabine in a PANC-1 (KRASG12D) NOD-SCID subcutaneous xenograft model. PANC-1 cells were subcutaneously implanted into NOD-SCID mice, and, following tumor establishment, animals were treated with vehicle control, gemcitabine (40 mg/kg, twice weekly), ABT-301 (100 mg/kg, once daily), or a combination of ABT-301 and gemcitabine according to the indicated dosing schedule. (A) longitudinal tumor growth curves depicting changes in tumor volume over the treatment period, with tumor volumes at the study endpoint (Day 29) summarized in the accompanying panel. Data are presented as mean±SEM, and each symbol represents an individual mouse. (B) changes in body weight during the treatment period, expressed as a percentage of initial body weight (mean±SEM). The dotted line indicates the predefined monitoring threshold. Statistical significance between groups is indicated as follows: *P<0.05, **P<0.01, ***P<0.001, and ****P<0.0001.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### DEFINITION

The terms used in this specification are generally within the scope of the present disclosure and the specific context of each term has its usual meaning in related fields. The specific terms used to describe the present disclosure in this specification will be described below or elsewhere in this specification, so as to help people in the industry understand the relevant description of the present invention. The same term has the same scope and meaning in the same context. In addition, there is more than one way to express the same thing; therefore, the terms discussed in this article may be replaced by alternative terms and synonyms, and whether a term is specified or discussed in this article does not have any special meaning. This article provides synonyms for certain terms, but the use of one or more synonyms does not mean that other synonyms are excluded.

As used herein, unless the context clearly indicates otherwise, “a” and “the” can also be interpreted as plural. Also, as used herein and in the claims, the terms “at least one” and “one or more” have the same meaning and include one, two, three, or more. The practice of the present disclosure will employ, unless otherwise indicated, conventional techniques of molecular biology, microbiology, recombinant DNA, and immunology, which are within the skill of the art. Such techniques are explained fully in the literature.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “effective amount” as referred to herein designate the quantity of a component which is sufficient to yield a desired response. For therapeutic purposes, the effective amount is also one in which any toxic or detrimental effects of the component are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms, or as milligrams per kilogram of body weight (mg/kg). Alternatively, the effective amount can be expressed in the concentration of the active component (e.g., the present agent ABT-301), such as molar concentration, mass concentration, volume concentration, molality, mole fraction, mass fraction and mixing ratio. Specifically, the term “therapeutically effective amount” used in connection with the agent described herein refers to the quantity of the agent, which is sufficient to alleviate or ameliorate the symptoms associated with the cancer in the subject. People who have ordinary skills could calculate the human equivalent dose (HED) for the medicament (such as the present agent ABT-301) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

As used herein, the term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alkyl)4 salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N+(C1-4 alkyl)4—salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein the term “ester” (or carboxylate, carboxylic acid ester or oxycarbonyl) represents a group of formula: —C(═O)OR, wherein R is an ester substituent, for example, a substituted or unsubstituted C1-10 alkyl group, a substituted or unsubstituted C3-20 heterocyclyl group, or a substituted or unsubstituted aryl group (typically a phenyl group). Examples of ester groups include, but are not limited to, —C(═O)OCH3, —C(═O)OCH2CH3, —C(═O)OC(CH3)3, and —C(═O)OPh.

As used herein, the term “solvate” may refer to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

As used herein, unless otherwise indicated, the term “treating” means reversing, alleviating, inhibiting the progress of, or preventing the disorder or condition to which such term applies, or one or more symptoms of such disorder or condition. The term “treatment”, unless otherwise indicated, refers to the act of treating as “treating” defined immediately above.

As used herein, the term “administered,” “administering” or “administration” are used interchangeably herein to refer either directly administering the pharmaceutical combination of the present disclosure, which comprises the (E)-N-hydroxy-3-(1-(phenylsulfonyl)-indolin-5-yl)-acrylamide and gemcitabine.

As used herein, the term “subject” or “patient” refers to an animal including the human species that is treatable with the pharmaceutical combination and/or method of the present disclosure. The term “subject” or “patient” intended to refer to both the male and female gender unless one gender is specifically indicated. Accordingly, the term “subject” or “patient” comprises any mammal which may benefit from treatment of cancer. Examples of a “subject” or “patient” include, but are not limited to, a human, or a non-human mammal including a chimpanzee, a monkey, a gorilla, a dog, a cat, a ferret, a rabbit, a guinea pig, a hamster, a gerbil, a cow, a horse, a pig, a sheep, a goat, a chicken, a turkey, a duck, a goose, a mouse, a rat, a zebrafish, an elephant, a lion, a tiger, a bear, a deer, a whale, a dolphin, or a seal. In an exemplary embodiment, the subject is a human.

As described herein, tumor responses were evaluated using Response Evaluation Criteria in Solid Tumors (RECIST) criteria. Complete response (CR) was defined as complete disappearance of measurable or evaluable tumor lesions; partial response (PR) was defined as reduction of the size of measurable tumor lesions by at least 30% (≥30%) compared to the original tumor size; stable disease (SD) was defined as reduction of the size of measurable tumor lesions by less than 30% (<30%) or enlargement of the size of measurable tumor lesions by less than 20% (<20%) compared to the original tumor size; and progressive disease (PD) was defined as growth of the tumor by more than 30% (>30%) in the tumor volume compared to the original tumor size or development of new lesions.

### PHARMACEUTICAL COMBINATION

In an embodiment, the present disclosure provides a pharmaceutical combination comprising a therapeutically effective amount of (E)-N-hydroxy-3-(1-(phenylsulfonyl)-indolin-5-yl)-acrylamide (alternatively known as ABT-301, or MPT0E028) or a pharmaceutically acceptable salt, ester, and/or solvate thereof, and a therapeutically effective amount of gemcitabine. ABT-301 is a selective inhibitor of class I and IIb histone deacetylases (HDACs), exhibiting greater inhibition of HDAC1, HDAC2, and HDAC3 compared to SAHA and Chidamide. Preclinical studies have demonstrated its ability to induce cell cycle arrest and apoptosis when used as monotherapy.

In some embodiments, ABT-301 is administered in a separate dosage form from the gemcitabine.

In some embodiments, ABT-301 is administered orally. Oral administration may be carried out in any pharmaceutically acceptable dosage form suitable for delivering ABT-301 to a subject in need thereof. Exemplary dosage forms include, but are not limited to, tablets, capsules, granules, powders, pellets, suspensions, emulsions, solutions, syrups, and reconstitutable formulations.

In some embodiments, ABT-301 is formulated as an immediate-release dosage form to facilitate rapid systemic absorption following oral intake. In other embodiments, ABT-301 may be provided in a modified-release formulation, including but not limited to sustained-release, controlled-release, delayed-release, or enteric-coated formulations, to optimize pharmacokinetic parameters such as Cmax, Tmax, bioavailability, or overall exposure.

The oral formulation may further comprise one or more pharmaceutically acceptable excipients, including but not limited to diluents, binders, disintegrants, lubricants, glidants, surfactants, stabilizers, buffering agents, or solubilizing agents. In certain embodiments, the formulation may include a vehicle comprising carboxymethylcellulose, polysorbates, sugars, polyols, or combinations thereof.

In some embodiments, ABT-301 is administered once daily, twice daily, or according to any suitable dosing schedule, including continuous or intermittent regimens. The dosage amount may vary depending on the subject's condition, body weight, age, disease severity, and concomitant therapies, and may range, for example, from about 1 mg/kg to about 200 mg/kg per administration, or any subrange therein.

In certain embodiments, oral administration of ABT-301 provides improved patient compliance compared to parenteral administration. In further embodiments, oral delivery enables convenient combination therapy, including co-administration with one or more additional therapeutic agents.

In some embodiments, the gemcitabine is administered parenterally, such as intravenously, intramuscularly, subcutaneously, or intraperitoneally. Intraperitoneal or intravenous administration may be performed by delivering gemcitabine directly into the peritoneal cavity using any pharmaceutically acceptable injectable formulation suitable for such administration.

In some embodiments, gemcitabine is provided as a sterile aqueous solution suitable for injection. The formulation may comprise gemcitabine or a pharmaceutically acceptable salt thereof, optionally in combination with one or more pharmaceutically acceptable carriers, diluents, buffering agents, tonicity-adjusting agents, stabilizers, or preservatives. Exemplary excipients include saline, phosphate-buffered saline (PBS), dextrose solutions, or other physiologically compatible aqueous vehicles.

In certain embodiments, gemcitabine is administered at a dosage ranging from about 1 mg/kg to about 200 mg/kg per administration, or any subrange therein. In some embodiments, gemcitabine is administered once weekly, twice weekly, or according to an intermittent dosing schedule, depending on the therapeutic protocol, disease condition, and tolerability profile of the subject.

Intraperitoneal administration may provide localized exposure within the peritoneal cavity and/or facilitate systemic absorption through the peritoneal membrane. In certain embodiments, intraperitoneal delivery allows controlled exposure levels while potentially reducing certain systemic toxicities associated with alternative routes of administration.

In some embodiments of the invention, intraperitoneal administration of gemcitabine is carried out in combination with oral administration of ABT-301, thereby providing a combination therapeutic regimen. The respective dosing schedules may be coordinated or staggered, and may be adjusted based on pharmacodynamic response, tolerability, or therapeutic outcome.

In some embodiments, the solvate of ABT-301 is IPA solvate, acetone solvate, ACN solvate, MeOH solvate, NMP solvate, THF solvate, EtOAc solvate, DMAc solvate, EtOH solvate, DCM solvate, DMSO solvate, 1-4-Dioxane solvate or MIBK solvate.

### PHARMACEUTICAL KIT

In an embodiment, the present disclosure provides a pharmaceutical kit for treating cancer in a subject, comprising the pharmaceutical combination described.

In certain embodiments, the pharmaceutical kit further comprises one or more additional therapeutic agents, optionally packaged separately or co-packaged within the same container, together with instructions for use. In some embodiments, the kit comprises a first container containing ABT-301 or a pharmaceutical composition comprising ABT-301, and a second container containing gemcitabine or a pharmaceutical composition comprising gemcitabine. The components may be provided in unit dosage forms suitable for repeated administration according to a predetermined dosing schedule.

In certain embodiments, the kit further comprises written instructions, labels, or prescribing information describing a method of treating cancer in a subject, including guidance regarding dosage, route of administration, dosing frequency, and treatment duration. In some embodiments, the instructions describe oral administration of ABT-301 and intraperitoneal or intravenous administration of gemcitabine, optionally according to an intermittent regimen.

In some embodiments, the kit is configured for combination therapy, wherein the pharmaceutical composition comprising ABT-301 and the composition comprising gemcitabine are administered sequentially, simultaneously, or within a defined treatment cycle. In certain embodiments, ABT-301 is administered once daily for five consecutive days followed by a drug-free interval, while gemcitabine is administered once or twice weekly; however, other dosing regimens may be employed depending on the therapeutic protocol.

In certain embodiments, the kit is intended for use in treating a proliferative disorder, including but not limited to pancreatic cancer, bladder cancer, colorectal cancer, breast cancer, prostate cancer, renal cancer, hepatocellular cancer, lung cancer, non-small cell lung cancer (NSCLC), ovarian cancer, cervix cancer, gastric cancer, esophageal cancer, neuroendocrine cancer, bone cancer, or head and neck cancer. In some embodiments, the cancer is a solid tumor. In other embodiments, the cancer is refractory, recurrent, metastatic, or resistant to prior chemotherapy.

In certain embodiments, administration of the components of the kit results in enhanced anti-tumor efficacy compared to administration of either agent alone. Without being bound by theory, the combination may provide synergistic, additive, or sensitizing effects on tumor cell growth inhibition, apoptosis induction, or tumor burden reduction.

In certain embodiments, the cancer is selected from the group consisting of pancreatic cancer, bladder cancer, colorectal cancer, breast cancer, prostate cancer, renal cancer, hepatocellular cancer, lung cancer, non-small cell lung cancer (NSCLC), ovarian cancer, cervix cancer, gastric cancer, esophageal cancer, neuroendocrine cancer, bone cancer or head and neck cancer.

### METHOD OF TREATMENT

The present disclosure provides a method of treating cancer, comprising administering ABT-301 or a pharmaceutically acceptable salt, ester, and/or solvate thereof, in combination with gemcitabine, according to a therapeutically effective regimen.

In some embodiments, ABT-301 is administered orally in the form of a tablet, capsule, granule, suspension, or other pharmaceutically acceptable dosage form. In certain embodiments, gemcitabine is administered parenterally, including but not limited to intraperitoneal, intravenous, intramuscular, or subcutaneous administration. In particular embodiments, ABT-301 is administered once daily for a defined treatment cycle, and gemcitabine is administered once or twice weekly; however, alternative dosing schedules may be employed based on therapeutic need.

In certain embodiments, the therapeutically effective amount of ABT-301 ranges from about 1 mg/kg to about 200 mg/kg per administration, or any subrange therein. In certain embodiments, the therapeutically effective amount of gemcitabine ranges from about 1 mg/kg to about 150 mg/kg per administration. The dosage and treatment duration may vary depending on the type and stage of cancer, the subject's physiological condition, prior treatment history, and tolerability.

In some embodiments, the two agents are administered sequentially, simultaneously, or within the same treatment cycle. In certain embodiments, the administration results in enhanced anti-tumor efficacy relative to administration of either ABT-301 or gemcitabine alone. Without wishing to be bound by theory, the combination therapy may result in synergistic or additive inhibition of tumor cell proliferation, increased induction of apoptosis, disruption of tumor cell cycle progression, or sensitization of tumor cells to nucleoside analog-mediated cytotoxicity.

In certain embodiments, the cancer treated according to the disclosed method is a solid tumor. In some embodiments, the cancer is metastatic, refractory, recurrent, or resistant to prior chemotherapeutic regimens. In particular embodiments, the cancer is pancreatic cancer.

In certain embodiments, the subject is a mammal. In some embodiments, the subject is a human. In other embodiments, the subject is a non-human mammal used for veterinary or research purposes, including but not limited to primates, companion animals, livestock animals, laboratory animals, or wildlife species as described herein. The disclosed method may therefore be applied in clinical, veterinary, or experimental settings.

### EXAMPLES

In this section, the contents of the present invention will be described in detail through the following examples. These examples are for illustration only, and those skilled in the art can easily think of various modifications and changes. As such, various embodiments of the present invention will be described in detail below, while the invention is not limited to said various embodiments listed in this specification.

### Example 1

### Preparation of Abt-301 Compound

ABT-301 compound can be synthesized/prepared via any suitable methods known in the art, such as the routes and methods disclosed in the U.S Pat. No. 8,846,748 B2.

### Example 2

### Evaluation of ABT-301 in Combination with Gemcitabine in a BxPC3 Pancreatic Tumor Xenograft Model

### In Vitro

In vitro studies demonstrated that the combination of ABT-301 with gemcitabine enhanced antitumor activity across multiple pancreatic ductal adenocarcinoma (PDAC) cell lines, including Panc-1, MIA PaCa-2, and BxPC-3. The combination exhibited greater growth inhibition than either agent alone, thereby supporting further in vivo evaluation.

### In Vivo

### Materials

The following materials were used:
Female BALB/c nude mice (6-7 weeks old; BioLASCO, Ilan, Taiwan)
ABT-301 powder (Lot: PJ05050-91-FP-P)
Carboxymethylcellulose (C4888, Sigma-Aldrich, St. Louis, MO, USA)
Tween 80 (P4780, Sigma-Aldrich, St. Louis, MO, USA)
Dextrose (D9434, Sigma-Aldrich, St. Louis, MO, USA)
BxPC3, pancreatic carcinoma cell line (BCRC 60283, Bioresource Collection and Research Center, Food Industry Research and Development Institute, Hsinchu, Taiwan)
RPMI-1640 medium (MT-10-040-CM, Corning)
Fetal bovine serum (MT-35-010-CV, Corning)
Matrigel™ (354234, Corning)
Syringe, 1 mL with needle 26G×1/2 inch (NIPRO Corp., Osaka, Japan)
Digital caliper (Mahr GmbH, Göttingen, Germany)
Animal Care and Tumor Implantation

Twenty-six female BALB/c nude mice were acclimatized in the feeding room of Taiwan Mouse Clinic for approximately one week under controlled conditions (temperature 20-26 °C; humidity 30-70%; 12-hour light/dark cycle). Food and water were provided ad libitum.

BxPC3 cells were cultured in RPMI medium supplemented with 10% fetal bovine serum at 37 °C in 5% CO₂. On the day of implantation, viable cells were counted using trypan blue exclusion. Cells were suspended in PBS and Matrigel™ at a 1:1 ratio. A total of 1×107 BxPC3 cells in 0.1 mL were subcutaneously injected into the left flank of each mouse using a 26G×1-inch needle.

Tumor dimensions were measured using a digital caliper, and tumor volume (mm³) was calculated using the formula: Volume=(length×width2)/2. When the mean tumor volume reached approximately 100 mm³, animals were randomized into treatment groups, and the test article was administered.

### Formulation of ABT-301

ABT-301 was formulated in a vehicle comprising 0.5% (w/v) carboxymethylcellulose and 0.1% (w/v) Tween 80 in 5% dextrose. ABT-301 (75 mg) was dissolved in 5 mL of vehicle using 50 ml centrifuge tube with vortex till a clear solution of 15 mg/mL ABT-301 was obtained. All dosing solutions were prepared at room temperature prior to administration.

### Treatment Regimen

Tumor-bearing mice were assigned to three groups:
Group 1 (Control): Vehicle administered via oral gavage.
Group 2 (Gemcitabine only): Gemcitabine administered intraperitoneally at predetermined dosing days (Day 4, 8, 11, 15, and 18).
Group 3 (Combination): Gemcitabine administered intraperitoneally at 75 mg/kg on Day 4, 8, 11, 15, and 18; and ABT-301 administered orally at 75 mg/kg twice daily for 21 consecutive days or at 75 mg/kg twice daily for 5 consecutive days followed by 2 days off per week, over a total period of 21 days.

Tumor volume and body weight were measured three times per week.

### Statistical Analysis

Data were expressed as mean ±standard error of the mean (SEM). Differences among groups were analyzed using two-way ANOVA. (GraphPad Prism 10, San Diego, CA, USA).

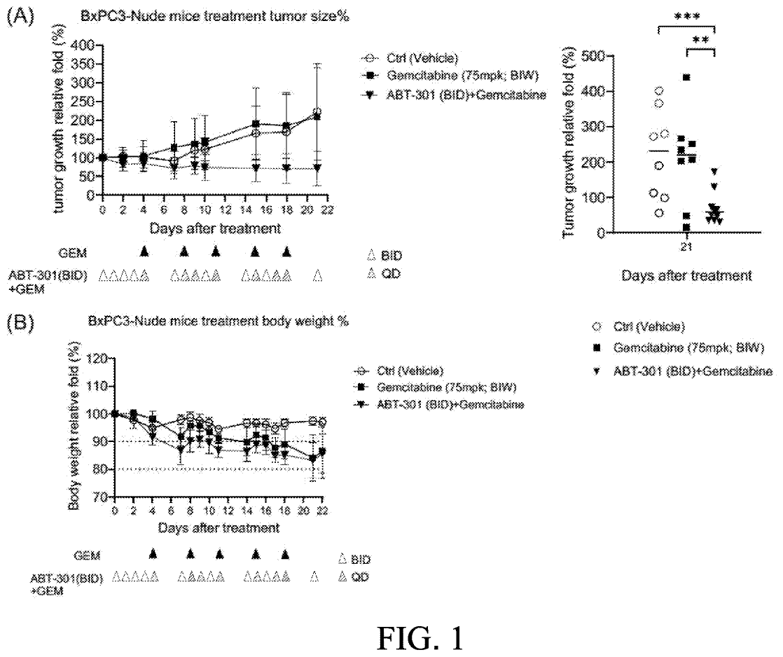

According to FIG. 1, ABT-301 in combination with gemcitabine demonstrated enhanced antitumor efficacy compared to gemcitabine alone in the BxPC3 xenograft model. At the end of the 21-day treatment period, the combination group exhibited a statistically significant reduction in tumor growth compared to the gemcitabine-only group (p<0.05). The tumor growth inhibition (TGI) rate was approximately 68% in the combination group, whereas gemcitabine alone produced approximately 6% TGI. A transient body weight reduction (>5%) was observed during the initial week of treatment and subsequently stabilized. The weight loss was attributed primarily to gemcitabine administration.

### Example 3

### Anti-Tumor Effect of ABT-301 in a PANC-1 (KRAS^{G12D}) Subcutaneous Xenograft Model in NOD-SCID Mice

This example evaluates the in vivo antitumor activity of ABT-301 administered orally in a PANC-1 pancreatic cancer xenograft model established in NOD-SCID mice, and further evaluates its therapeutic effect in combination with gemcitabine.
Material
Product name: ABT-301
Lot No.: PJ05050-103-FP-P
Manufacturer: ANBOGEN THERAPEUTICS, INC.
Major ingredient: Histone deacetylase (HDAC) inhibitor
Physical form: White powder
Sterilization: Not sterilized
Storage conditions: Room temperature
Gemcitabine was obtained from a commercial source and prepared according to standard laboratory procedures suitable for intraperitoneal administration.
Experimental Animals
Species/Strain: NOD-SCID mice
Sex: Male
Age at dosing: Approximately 7-9 weeks
Body weight range: 16-25 g
Source: BioLASCO, Taiwan
Total number: 64 mice (8 mice per group)
Animals were housed in individually ventilated cages (IVC) under the following controlled conditions:
Temperature: 20-26 °C
Relative humidity: 30-70%
Light/dark cycle: 12 h/12 h
Diet: Sterilized Lab Diet 5001 (ad libitum)
Water: RO water (ad libitum)
Bedding: Sterilized natural corn cob bedding (changed weekly)

Animal care was performed daily. Pain and distress were assessed twice weekly.

Since the test article is an anti-tumor therapeutic, NOD-SCID immunodeficient mice were selected as the experimental model based on literature references.

### Tumor Implantation

PANC-1 (KRASG12D) human pancreatic cancer cells were used for tumor implantation. A total of 5×10⁶ PANC-1 cells were suspended in appropriate medium and subcutaneously implanted into mice. During tumor cell inoculation, mice were anesthetized briefly with 4% isoflurane at a flow rate of approximately 400 mL/min.

Tumor dimensions were measured using calipers twice weekly. Tumor volume (mm³) was calculated using the following formula: Volume=(Length×Width²)/2

When mean tumor volume reached approximately 120 mm³, animals were randomized into treatment groups.

Study Design and Treatment Regimen
The total study duration was approximately 7-9 weeks, including:
Tumor establishment: 2-3 weeks
Dosing period: 4 weeks
Post-dosing observation: 1-2 weeks

Four treatment groups were established:
Group 1 (Vehicle Control): Vehicle administered orally once daily for five consecutive days followed by two days without dosing each week.
Group 2 (Gemcitabine Monotherapy): Gemcitabine administered intraperitoneally at 40 mg/kg (40 mpk), twice weekly.
Group 3 (ABT-301 Monotherapy): ABT-301 administered orally once daily for five consecutive days followed by two days without dosing each week.
Group 4 (Combination Therapy): ABT-301 administered orally once daily for five consecutive days followed by two days without dosing each week, in combination with gemcitabine administered intraperitoneally at 40 mg/kg twice weekly.

Body weight was recorded twice weekly. Clinical signs of toxicity or discomfort were monitored daily.

### Humane Endpoints

Animals were euthanized if any of the following criteria were met:
Tumor volume≥4000 mm³
Body weight loss≥20%
Severe ulceration, complications, or non-recoverable distress

Euthanasia was performed using carbon dioxide according to institutional procedures “WI-AF-02-04”.

### Statistical Analysis

Longitudinal tumor growth curves were analyzed using two-way ANOVA with treatment group and time as factors, followed by multiple comparison testing where appropriate. Tumor volumes at Day 29 were analyzed using Welch's t-test to account for unequal variances. Data are expressed as mean±SEM. Statistical significance was defined as p<0.05.

### Results

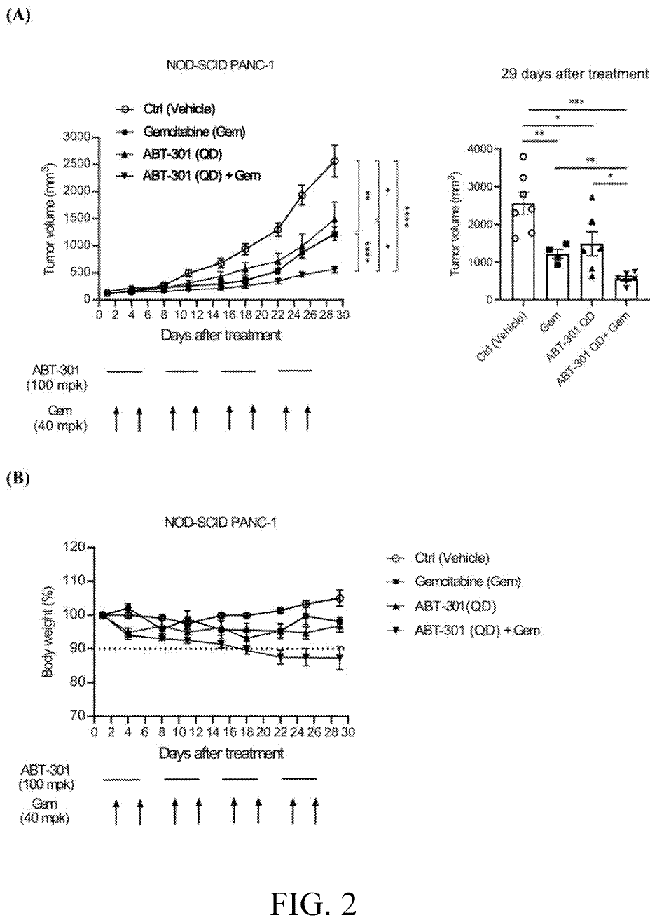

According to FIG. 2, In the PANC-1 (KRASΛG12DΛ) xenograft model, vehicle-treated tumors exhibited rapid and sustained growth throughout the study period. Gemcitabine monotherapy significantly suppressed tumor growth compared with vehicle controls, resulting in a marked reduction in tumor volume at the study endpoint (Day 29).

ABT-301 monotherapy also significantly inhibited tumor growth relative to the vehicle group, demonstrating in vivo antitumor activity in this KRAS-mutant model. Although the magnitude of tumor suppression observed with ABT-301 alone was less pronounced than that of gemcitabine, tumor volumes remained substantially smaller than those of vehicle controls at study termination.

Combination treatment with ABT-301 and gemcitabine produced the greatest tumor growth inhibition among all treatment groups. The combination yielded the lowest tumor volumes at Day 29 and demonstrated enhanced tumor control compared to either monotherapy, consistent with additive antitumor efficacy. Throughout the treatment period, body weight was largely maintained in the vehicle, gemcitabine, and ABT-301 monotherapy groups. A moderate and progressive decrease in body weight was observed in the combination group; however, body weight remained above predefined humane endpoints.

In summary, the effect of the present disclosure is that the specific combination of ABT-301 and gemcitabine can exhibit significantly stronger anti-tumor effects compared to treatment using either agent alone. This indicates the potential clinical utility of ABT-301 as a sensitizing agent in the treatment of cancer when used in combination with chemo drugs.

The specific embodiments of the present invention have been disclosed, but it is not intended to limit the present invention. Those with ordinary knowledge in the technical field to which the present invention belongs are capable of understanding. And in the case of deviating from the principle and spirit of the present invention, various changes and modifications can be made to it, so the scope of protection of the present invention should be based on those defined in the scope of the accompanying patent application.

While the present disclosure has been described by means of specific embodiments, numerous modifications and variations could be made thereto by those skilled in the art without departing from the scope and spirit of the present disclosure set forth in the claims.

What is claimed is:

1. A pharmaceutical combination comprising
a therapeutically effective amount of (E)-N-hydroxy-3-(1-(phenylsulfonyl)-indolin-5-yl)-acrylamide or a pharmaceutically acceptable salt, ester, and/or solvate thereof; and
a therapeutically effective amount of gemcitabine.

2. The pharmaceutical combination of claim 1, wherein the (E)-N-hydroxy-3-(1-(phenylsulfonyl)-indolin-5-yl)-acrylamide is formulated in a separate dosage form from the gemcitabine.

3. The pharmaceutical combination of claim 2, wherein the (E)-N-hydroxy-3-(1-(phenylsulfonyl)-indolin-5-yl)-acrylamide is formulated for oral administration.

4. The pharmaceutical combination of claim 2, wherein the gemcitabine is formulated for parenteral administration, intravenous, intramuscular, subcutaneous, or intraperitoneal administration.

5. The pharmaceutical combination of claim 1, wherein the solvate of (E)-N-hydroxy-3-(1-(phenylsulfonyl)-indolin-5-yl)-acrylamide is IPA solvate, acetone solvate, ACN solvate, MeOH solvate, NMP solvate, THF solvate, EtOAc solvate, DMAc solvate, EtOH solvate, DCM solvate, DMSO solvate, 1-4-Dioxane solvate or MIBK solvate.

6. A pharmaceutical kit for treating cancer in a subject, comprising the pharmaceutical combination of claim 1.

7. The pharmaceutical kit of claim 6, wherein the cancer is selected from the group consisting of pancreatic cancer, bladder cancer, colorectal cancer, breast cancer, prostate cancer, renal cancer, hepatocellular cancer, lung cancer, non-small cell lung cancer (NSCLC), ovarian cancer, cervix cancer, gastric cancer, esophageal cancer, neuroendocrine cancer, bone cancer or head and neck cancer.

8. A method for treating cancer in a subject, comprising administering to the subject a therapeutically effective amount of (E)-N-hydroxy-3-(1-(phenylsulfonyl)-indolin-5-yl)-acrylamide or a pharmaceutically acceptable salt, ester, and/or solvate thereof; and
administering to the subject a therapeutically effective amount of gemcitabine.

9. The method of claim 8, wherein the (E)-N-hydroxy-3-(1-(phenylsulfonyl)-indolin-5-yl)-acrylamide and the gemcitabine are administered sequentially, simultaneously, or within the same treatment cycle.

10. The method of claim 8, wherein the cancer is selected from the group consisting of pancreatic cancer, bladder cancer, colorectal cancer, breast cancer, prostate cancer, renal cancer, hepatocellular cancer, lung cancer, non-small cell lung cancer (NSCLC), ovarian cancer, cervix cancer, gastric cancer, esophageal cancer, neuroendocrine cancer, bone cancer or head and neck cancer.

11. The method of claim 8, wherein the subject is a human.

12. The method of claim 8, wherein the subject is a non-human mammal.
